(19) United States
(12) Patent Application Publication
(10) Pub. No.: US 2025/0328831 A1
(43) Pub. Date: Oct. 23, 2025

(54) MATCHING METHOD, MATCHING SYSTEM, AND STORAGE MEDIUM

(71) Applicant: TOYOTA JIDOSHA KABUSHIKI KAISHA, Toyota-shi (JP)

(72) Inventors: Satoshi TOYOSHIMA, Toyota-shi (JP); Shiro ODA, Anjo-shi (JP); Takeshi MATSUI, Nisshin-shi (JP)

(73) Assignee: TOYOTA JIDOSHA KABUSHIKI KAISHA, Toyota-shi (JP)

(21) Appl. No.: 18/983,620

(22) Filed: Dec. 17, 2024

(30) Foreign Application Priority Data
Apr. 19, 2024 (JP) 2024-068366

(51) Int. Cl. G06Q 10/0631; G06Q 10/0639

(52) U.S. Cl. CPC G06Q 10/063112; G06Q 10/06393

(57) ABSTRACT

The matching method according to the present embodiment is a matching method for matching a robot introduction-desired person and a robot supplier using a computer, wherein request information regarding the robot introduction from the introduction-desired person is acquired, supply information regarding a robot that can be supplied by the supplier is acquired, matching is performed based on the request information and the supply information, and a matching result is notified to at least one of the introduction-desired person and the supplier. The matching device may generate the matching using a machine learning model such as deep learning.

### CROSS-REFERENCE TO RELATED APPLICATION

This application claims priority to Japanese Patent Application No. 2024-068366 filed on Apr. 19, 2024, incorporated herein by reference in its entirety.

### BACKGROUND

### 1. Technical Field

The present disclosure relates to a matching method, a matching system, and a storage medium.

### 2. Description of Related Art

Japanese Unexamined Patent Application Publication No. 2023-81320 (JP 2023-81320 A) discloses a disclosure system that manages an autonomous vehicle in a hospital. In a hospital, an autonomous vehicle carries medical supplies, drugs, linen, specimens, meals, and the like.

### SUMMARY

Incidentally, when a robot such as an autonomous vehicle is introduced into a facility, an introduction-desired person who wishes to introduce the robot investigates what type of robot should be introduced. However, when services (also called tasks) to be executed by the robot are diverse, there is a problem that the investigation becomes complicated.

A matching method relating to the present aspect is

a matching method that matches an introduction-desired person of a robot and a supplier of the robot by using a computer, the matching method including:
obtaining request information related to a robot introduction from the introduction-desired person,
obtaining supply information related to the robot, the supply information being suppliable by the supplier,
performing matching based on the request information and the supply information, and notifying a matching result to at least one of the introduction-desired person and the supplier.

According to the present disclosure, it is possible to provide a matching method, a matching system, and a storage medium capable of appropriately matching an introduction-desired person and a supplier of a robot.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a schematic diagram illustrating an overall configuration of a matching system according to the present embodiment;

FIG. 2 is a block diagram illustrating an overall configuration of a matching system;

FIG. 3 is a table illustrating an example of request information;

FIG. 4 is a table illustrating an exemplary supply information; and

FIG. 5 is a flowchart illustrating a matching method.

### DETAILED DESCRIPTION OF EMBODIMENTS

Hereinafter, the present disclosure will be described through embodiments of the disclosure. However, the disclosure according to the claims is not limited to the following embodiments. Moreover, all of the configurations described in the embodiments are not necessarily indispensable as means for solving the issue.

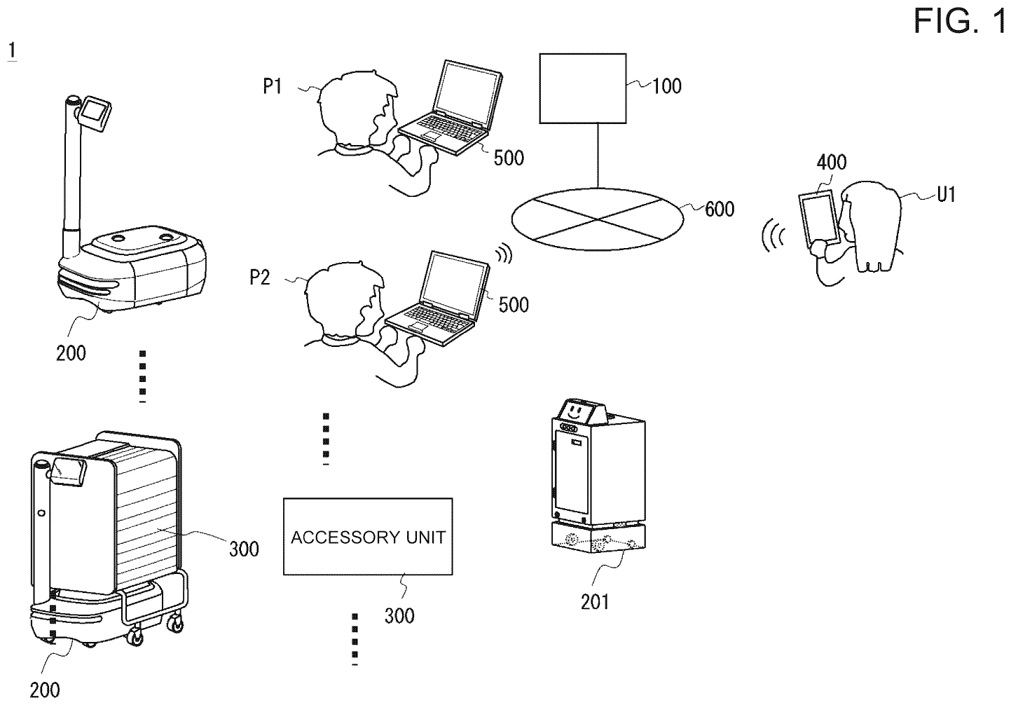

Referring to FIG. 1, a matching system according to the present embodiment will be described. FIG. 1 is a schematic diagram illustrating an overall configuration of a matching system. The matching system 1 includes a matching device 100, an applicant terminal 400, and a supplier terminal 500. The matching device 100, the applicant terminal 400, and the supplier terminal 500 are connected via a network 600.

The applicant terminal 400 is a tablet computer, a smartphone, a personal computer, or the like. The applicant terminal 400 may be any information processing apparatus capable of wirelessly or wirelessly communicating. The applicant U1 is a robot introduction-desired person who desires to introduce the robots 200, 201. For example, the applicant U1 is an owner, an administrator, an area-responsible person, or the like of a facility, and desires to introduce the robots 200 and 201 that execute various services (also referred to as tasks). The applicant U1 operates the applicant terminal 400 to introduce the robots 200 and 201 to the facilities. The applicant U1 uses the applicant terminal 400 to enter information required for introduction.

The supplier terminal 500 is used by the supplier P1, P2. The supplier terminal 500 is an information processing apparatus such as a tablet computer, a smartphone, or a personal computer. The supplier P1, P2 operates the supplier terminal 500 to enter supply information related to the supplier-capable robots. Although only two supplier P1, P2 are shown in FIG. 1, there may be three or more suppliers.

The matching device 100 is used by a matching vendor (not shown). Matching agents are operators and administrators of matching websites that match the applicant U1 with the supplier P1. The matching device 100 is an information processing device such as a tablet computer, a smartphone, or a personal computer. The matching device 100 performs a matching process of matching the applicant U1 and the supplier P1, P2. That is, the matching device 100 selects a supplier P1 according to the desire of the applicant U1 from among the plurality of suppliers P1, P2. The matching device 100 transmits the selected supplier P1 to the applicant U1. Alternatively, the matching device 100 transmits the applicant U1 to the selected supplier P1. The matching device 100 may select two or more supplier P1 and transmit respective data.

The matching device 100, the applicant terminal 400, and the supplier terminal 500 can be implemented as, for example, a program-executable device such as a central processing unit (CPU: Central Processing Unit). Various functions can also be realized by the program. The matching device 100, the applicant terminal 400, and the supplier terminal 500 have a communication interface for connecting to the network 600.

The supplier P1 selected by the matching device 100 supplies the robots 200 and 201 to the applicant U1 based on the introduction demand from the applicant U1. The supplier P1, P2 is, for example, a sales company, a manufacturing company, a sales agent, an intermediary, a maintenance company, a trading company, or an individual of the robot 200. The supplier P1, P2 may also provide a plurality of types of robots 200 and 201. The robotic delivery by the supplier P1, P2 may take the form of sales, leasing, renting, etc. The supplier P1, P2 may provide an accessory unit 300, which will be described later.

The robot 200 and the robot 201 are, for example, autonomous mobile robots, and execute services such as transportation, cleaning, security, and guidance. The robots 200 and 201 autonomously move medical welfare facilities such as hospitals, rehabilitation centers, nursing facilities, and residential facilities for the elderly. The robots 200 and 201 can also be used in a commercial facility such as a shopping mall. The robots 200 and 201 include wheels, a chassis, a motor, a sensor, a battery, a controller, and the like.

The robot 200 executes a service by using a combination of the accessory units 300. Different accessory units 300 are provided depending on the service. The robot 201 is a transfer robot that independently executes a transfer service. For example, the robot 201 includes a storage unit or the like that stores a conveyed object, and conveys the conveyed object without using the accessory unit 300. The robot 200 and the robot 201 carry a medicine, a sample, a medical record, a medical device, and the like.

The accessory unit 300 is used in combination with the robot 200 to execute each service. For example, when the robot 200 executes a transport service for transporting a conveyed object, the accessory unit 300 becomes a transport unit transported by the robot 200. The accessory unit 300 for transportation is a wagon or a cart with wheels, and a transported object can be mounted in the wagon or cart. The bogie portion of the robot 200 serves as a stage on which a wagon or the like is mounted.

The robot 200 has a lifting function for lifting the accessory unit 300. When the bogie portion of the robot 200 enters the lower side of the accessory unit 300, an elevating stage or the like lifts the accessory unit 300. Thus, the robot 200 mounts the accessory unit 300. The robot 200 can convey the conveyed object accommodated in the accessory unit 300 to the destination. The robot 200 can execute the conveyance service by mounting the accessory unit 300. The accessory unit 300 for transportation is used for setting and clearing the table in a restaurant, a medical welfare facility, or the like, or for a lower table. The transport unit may be used for transporting parts in a factory, transporting samples in a hospital, transporting linen in a hotel, and the like.

In addition, the robot 200 can perform a service other than the conveyance service together with the other accessory units 300. Examples of other services include cleaning services, security services, and guidance services. Accordingly, the accessory unit 300 for transportation includes an accessory unit for cleaning, an accessory unit 300 for security, and an accessory unit 300 for guidance. The robot 200 mounts any one of the accessory units. That is, the robot 200 can attach and detach the accessory unit 300. The robot 200 is equipped with an accessory unit 300 corresponding to a service to be executed.

The accessory unit 300 for cleaning is a unit for cleaning a facility. The accessory unit 300 for cleaning includes a cleaner for sucking dust and the like. Alternatively, the cleaning accessory unit 300 includes a brush, a pad for wiping the floor, a mop, and the like. When the robot 200 moves with the accessory unit 300 for cleaning attached thereto, the floor surface is cleaned. That is, in the region where the robot 200 has moved, the floor surface is cleaned. The robot 200 can execute the cleaning service by mounting the accessory unit 300 for cleaning. The accessory unit 300 for cleaning may also be a remotely controllable robotic cleaner. In this case, the robot 200 performs the cleaning service by remotely controlling the accessory unit 300 for cleaning by wireless communication.

The accessory unit 300 for security is a unit for security of a facility. The accessory unit 300 for security includes, for example, various sensors for detecting an intruder or an abnormality. For example, the security accessory unit 300 includes a camera, an infrared camera, a ranging sensor, an optical sensor, a thermal sensor, and a smoke sensor as sensors. A lighting device for illuminating an intruder or an abnormal portion may be provided. The accessory unit 300 may have an alarm function or the like for notifying when an abnormality is detected. The robot 200 can execute the security service by wearing the accessory unit 300 for security.

The guide accessory unit 300 is a unit for guiding a facility. For example, the accessory unit 300 for guidance guides a visitor of the facility to a route to the destination. The guiding accessory unit 300 has an input device for the visitor to input the destination. The input device includes a touch panel and buttons. The input device also includes a microphone for voice input. The robot 200 moves to a destination to guide the visitor. The guide accessory unit 300 may include a display device for displaying a route and a speaker for outputting a voice of the route.

As described above, when the attached unit 300 is used in combination with the robot 200, a plurality of different services can be executed by the robot 200. That is, the robot 200 executes a service corresponding to the attached unit 300 mounted thereon. Of course, the types of robots that can be introduced are not limited to two types. For example, the type of the robot may be one type or three or more types. In addition, a robot that executes a service other than the conveyance service may be introduced. A dedicated robot that performs various services such as a cleaning service, a security service, and a guidance service may be introduced.

Depending on the supplier P1, P2, the types of robots 200, 201 or accessory units 300 that can be supplied are different. The supplier P2 cannot provide the robots 200,201 or accessory units that can be supplied by the supplier P1. For example, the supplier P1 may share the robot 201 and the security accessory unit 300, but the supplier P2 may not share the robot 201 and the security accessory unit 300. That is, the supplier P2 does not handle the security accessory unit 300. In addition, the supplier P1 may not be able to supply the robots 200,201 or the attached units that can be supplied by the supplier P2. For example, the supplier P2 can provide an accessory unit 300 for cleaning, but the supplier P2 cannot provide an accessory unit 300 for cleaning.

Alternatively, when there are a plurality of types of robots or accessory units 300 that perform the same service, the supplier P1, P2 may not be able to supply some of the robots 200 or accessory units 300.

The matching device 100 supports the introduction of the robots for the applicant U1 by selecting the supplier P1 according to the request of the applicant U1. Specifically, the matching device 100 presents to the applicant U1 the supplier P1 that can provide the robots 200 and 201 that satisfy the desire of the applicant U1. Accordingly, an appropriate supplier P1 can be selected from among a large number of supplier P1. Therefore, the matching device 100 can appropriately support the introduction of the robots to the facilities of the applicant U1.

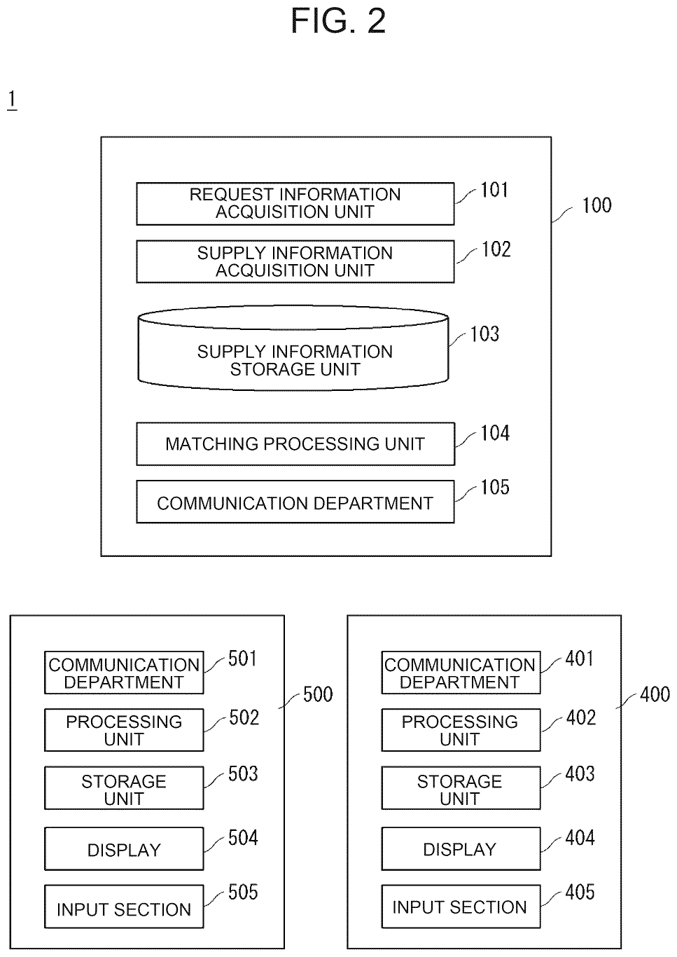

Hereinafter, the processing in the matching system 1 will be described with reference to FIG. 2. FIG. 2 is a block diagram illustrating a configuration of the matching system 1. Note that the matching device 100, the applicant terminal 400, and the supplier terminal 500 are not limited to a physically single apparatus, and may be distributed among a plurality of devices.

As described above, the applicant terminal 400 is an information processing terminal such as a smartphone. The applicant terminal 400 includes a communication unit 401, a processing unit 402, a storage unit 403, a display unit 404, and an input unit 405. The communication unit 401 has a communication interface such as a Wi-Fi (registered trademark) router. The communication unit 401 transmits and receives data to and from the communication unit 105 of the matching device 100. The communication unit 401 transmits request information to be described later to the matching device 100.

The processing unit 402 includes a processor and the like. The storage unit 403 includes a storage device such as a memory, a hard disk drive, or an SSD (Solid State Drive). The storage unit 403 stores programs, various settings, and the like. The processing unit 402 executes a program recorded in the storage unit 403 to perform various kinds of processing.

The display unit 404 includes a display device that performs display on an applicant U1. The display unit 404 displays a processing result of the processing unit 402, data received from the matching device 100, and the like. The input unit 405 includes an input device such as a touch panel, a keyboard, and a mouse. The input unit 405 receives a data input from the applicant U1. In other words, the applicant U1 operates the touch panel or the like to input data. The applicant U1 operates the input unit 405 to input the request information. Further, the input unit 405 may include a microphone or the like for voice input.

As described above, the supplier terminal 500 is an information processing terminal such as a smartphone or a personal computer. The supplier terminal 500 includes a communication unit 501, a processing unit 502, a storage unit 503, a display unit 504, and an input unit 505. The communication unit 401 has a communication interface such as a Wi-Fi (registered trademark) router. The communication unit 401 transmits and receives data to and from the communication unit 105 of the matching device 100. The communication unit 401 transmits supply information to be described later to the matching device 100. The configurations and functions of the communication unit 501, the processing unit 502, the storage unit 503, the display unit 504, and the input unit 505 are the same as those of the communication unit 501, the processing unit 502, the storage unit 503, the display unit 504, and the input unit 505, and thus description thereof is omitted as appropriate. The storage unit 403 and the storage unit 503 are each an example of a storage medium.

The matching device 100 includes a request information acquisition unit 101, a supply information acquisition unit 102, a matching processing unit 104, and a communication unit 105. The matching device 100 is a server computer or the like that collects data from the applicant terminal 400 and the supplier terminal 500. The processor of the matching device 100 executes the program stored in the memory to execute the following processing.

The applicant U1 operates the input unit 405 to input a request information for introduction of the robot. The request information acquisition unit 101 acquires the request information transmitted from the applicant terminal 400. For example, the applicant U1 operates the input unit 405 of the applicant terminal 400 to access a web page or the like of the matching device 100. The request information acquisition unit 101 transmits data for displaying an input screen of the request information to the applicant terminal 400.

The display unit 404 displays a screen for inputting information related to the robot to be introduced. The applicant U1 operates the input unit 405 to input the request information regarding the robot to be introduced. The input of request information in the input unit 405 may be input by response to text input, touch panel input, selection input, speech input, speech AI (Artificial Intelligence), etc. The communication unit 401 transmits a signal including the request information to the matching device 100. When the communication unit 105 of the matching device 100 receives the signal, the request information acquisition unit 101 extracts the request information.

The request information may include information indicating a robot to be introduced. For example, the request information is information indicating a model number, a type, and the like of the robot 200 in the facility. Alternatively, the request information may include information about a service executed by the robot 200 in the facility. The request information may include the number of robots to be introduced. The request information may include information such as a type of service, a frequency, a service range, and a service time. The request information may include information about the accessory unit 300 used for the service.

The request information may include information about a facility to be introduced. For example, information about the type, size, area, rank, and facility name of the facility may be included. The request information acquisition unit 101 acquires the request information transmitted from the applicant terminal 400. The request information may include information on the cost, the number of units, and the like. The request information may be a facility name of a facility, an address of a web page, or the like. In this case, the request information acquisition unit 101 acquires the information on the facility from the web page, and sets the information as the request information.

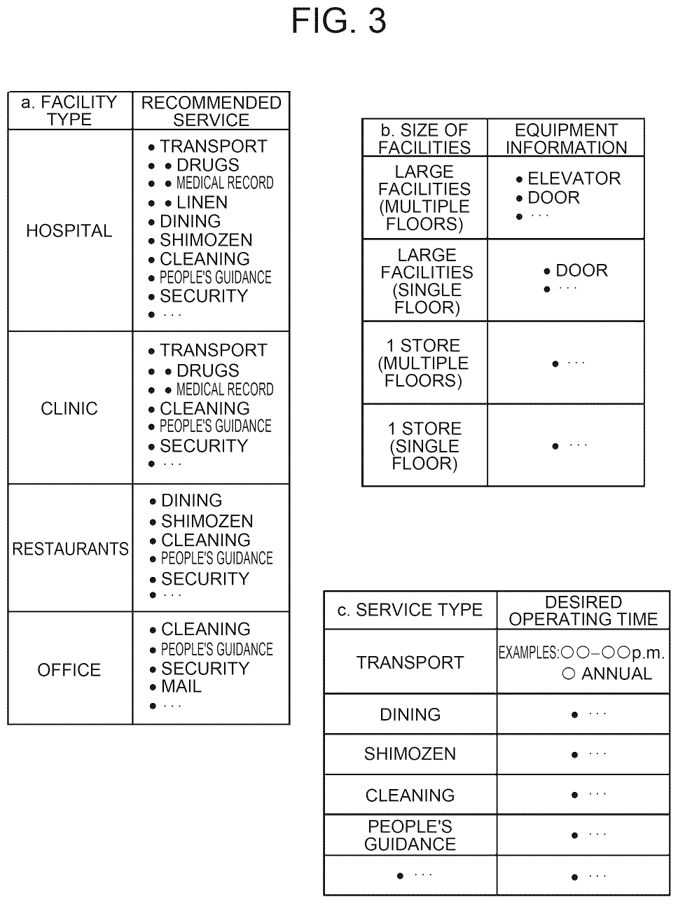

FIG. 3 is a diagram illustrating a table of request information. In FIG. 3, a table a, a table b, and a table c indicating request information are illustrated. Of course, the request information is not limited to the data of the table shown in FIG. 3. For example, data other than the data illustrated in FIG. 3 may be added to the request information. Alternatively, at least part of the data illustrated in FIG. 3 may not be included in the request information.

In the table a, the request information is the type of the facility in which the robot is introduced. For example, the applicant U1 selects and enters any one of a hospital, a clinic, a restaurant, and an office as the facility types. Then, the request information acquisition unit 101 acquires the facility type or the recommended service according to the facility type as the request information. Alternatively, the applicant U1 may enter a service to be executed as the request information.

In the table b, the request information is the facility size of the facility in which the robot is introduced. For example, the applicant U1 selects and enters one of a large facility having a plurality of stories, a large facility having a single story, and a single store having a single story on a plurality of stories. Further, the applicant U1 may enter, as the requested information, the facility information regarding the facility of the facility. For example, the applicant U1 may enter the number of elevators and the number of doors as request information. The request information may include the size of the passageway, the size of the elevator, and the size of the door. If the size is such that the robot cannot pass through, the matching device 100 makes the matching result bad.

In the table c, the request information is input as a service type indicating the type of the service to be executed in the facility. For example, the applicant U1 selects and inputs the type of the service to be executed in the facility as the request information. Further, the applicant U1 may enter a desired operating period of the service, and the like.

The supplier P1, P2 operates the input unit 505 to input supply information related to the robot supply. For example, the supplier P1 enters information about the robots and accessory units that it is handling. Similarly, the supplier P2 enters information about the robots and accessory units that it is handling. The supply information acquisition unit 102 acquires the supply information transmitted from the supplier terminal 500. For example, the supplier P1 operates the inputter 505 of the supplier terminal 500 to access a web page or the like of the matching device 100. The supply information acquisition unit 102 transmits data for displaying an input screen of the supply information to the supplier terminal 500.

The display unit 504 displays a screen for inputting information related to a suppliable robot. The supplier P1 operates the input unit 505 to input supply information related to a suppliable robot. The input of supply information in the supplier terminal 500 may be input by response to text entry, touch panel entry, selection entry, speech entry, speech AI (Artificial Intelligence), etc. The communication unit 501 transmits a signal including the supply information to the matching device 100. When the communication unit 105 of the matching device 100 receives the signal, the supply information acquisition unit 102 extracts the supply information. Then, upon acquiring supply information from a plurality of suppliers, the supply information acquisition unit 102 writes the supply information into the supply information storage unit 103.

The supply information storage unit 103 stores the supply information acquired by the supply information acquisition unit 102. The supply information storage unit 103 stores supply information acquired from a plurality of supplier P1, P2 as a data base. That is, the supply information storage unit 103 stores supply information related to a suppliable robot for each supplier. The supply information storage unit 103 stores information such as a contact address of each supplier P1, P2. In the case of performing matching, the matching device 100 may read the supply information stored in the supply information storage unit 103 in advance.

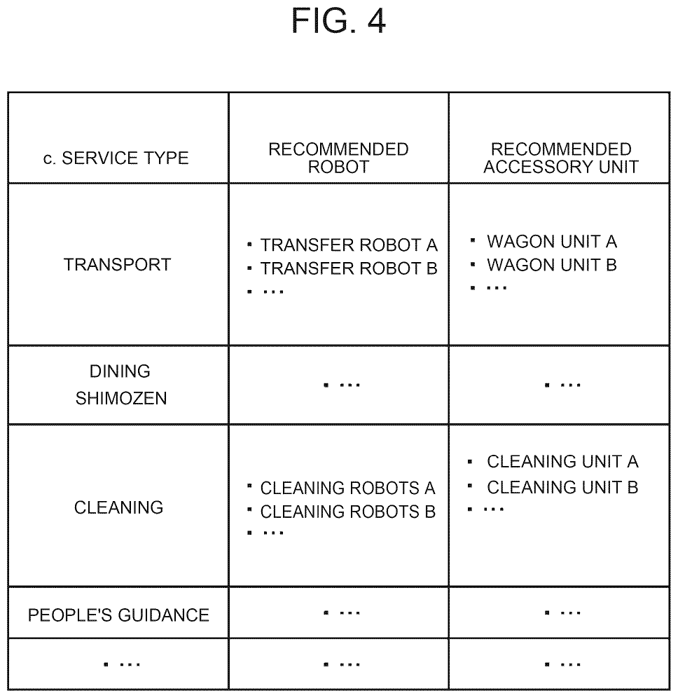

FIG. 4 is a diagram illustrating a table of supply information. Of course, the supply information is not limited to the data shown in the table shown in FIG. 4. For example, data other than the data illustrated in FIG. 4 may be added to the supply information. Alternatively, at least part of the data illustrated in FIG. 4 may not be included in the supply information.

The supplier P1 enters the service-type as supply information about the supplier-capable robots. Then, the supplier P1 inputs the model number and the like of the robots that can execute the service as the supply information. The supplier P1 enters the model number of the attached unit as supply information for performing the servicing. The supplier P1 may enter information on the performance, size, and the like of the robots and the attached units as the supply information. The supplier P1 may enter the cost of the robots or accessory units as the supply information. The cost includes the introduction cost such as the initial cost and the operation cost such as the running cost and the maintenance cost. Also, the cost may be a monthly cost such as a subscription or a yearly cost. The supply information may include an introduction cost for each robot or for each attached unit.

The supply information may include information about the operating time, operating area, operating range, and operating speed at which each robot performs a service. For example, the supply information may include information regarding the cleaning area per hour, the security area, the guidance distance, or the transport distance. Alternatively, the movable range may include information regarding the width of the passage, information indicating whether or not the vehicle is movable by the elevator, or information indicating whether or not the vehicle can be used outdoors.

The supply information may include detailed information about the executable service content. For example, when the robot is a transfer robot, the supply information may include information indicating a conveyable object. For example, it is assumed that there is a plurality of types of transported items, such as a medicine, a medical record, and linen. In this case, the supply information may include information indicating whether or not each conveyed object can be conveyed. The supply information may include information regarding the weight and size of the transportable object. For example, since the transfer robot A can lock the storage portion of the conveyed object, it transports important conveyed objects such as medicines and medical records. The transfer robot B is a wheelchair-type robot or a stretcher-type robot that conveys a patient. The wagon unit A is an accessory unit for carrying linen. The wagon unit B is an accessory unit 300 for transporting corrugated cardboard, fixtures, and the like. The supply information may include such information.

The supply information may include information about a cleanable floor surface. For example, the supply information includes information indicating that the cleaning unit A is a unit for carpet cleaning and the cleaning unit B is a unit for tile cleaning.

The matching processing unit 104 performs matching between the request information and the supply information. The matching processing unit 104 compares the request information with the supply information, and selects a supplier capable of executing the service indicated by the request information. That is, when there is a service that the robot or the attached unit cannot execute, the matching processing unit 104 excludes the supplier. For example, if applicant U1 enters the cleaning service as the requested information, it excludes a supplier who can only provide the robots that perform the transportation service. The matching processing unit 104 compares the request information with the supply information to determine whether or not the service is available. The matching processing unit 104 selects a supplier so that all services can be executed.

The supply information storage unit 103 stores supply information of a plurality of supplier P1, P2. The matching processing unit 104 sequentially reads the supply information stored in the supply information storage unit 103 and compares it with the request information. Then, the matching processing unit 104 outputs a matching result for each supplier. The matching result may be output in two stages indicating whether or not matching is possible, or may be indicated in multiple stages as a matching score.

In a case where all services cannot be executed by a robot supplied by one supplier, the matching processing unit 104 may combine two suppliers. In this case, the matching processing unit 104 selects a combination of two suppliers. Alternatively, the matching device 100 may transmit data prompting the applicant terminal 400 to input again. For example, since there is no matched supplier, the matching device 100 transmits a message for changing the request information to the applicant terminal 400.

When there are two or more suppliers capable of supplying robots capable of executing all services, the matching processing unit 104 may select two or more suppliers. Alternatively, among the two or more suppliers, the supplier may be selected according to the request information of the applicant U1. For example, when the applicant U1 desires to reduce the number of robots to be introduced, a supplier may be selected according to the desire. For example, the matching processing unit 104 selects a supplier P1 when there is a supplier P1 that supplies one robot capable of performing all services and a supplier P2 that supplies three robots capable of performing all services.

Alternatively, in a case where there is a desire to introduce at a lower cost, the matching processing unit 104 selects a supplier according to the desire. For example, if the cost for introducing the robot of the supplier P1 is less expensive than the cost for introducing the robot of the supplier P2, the matching processing unit 104 selects the supplier P2. When there is a plurality of requests, the requester may set a priority order for the requests.

The matching processing unit 104 may estimate the type and the number of robots to be introduced from the request information. In the supply information, it is assumed that the operation amount of the service is set for each robot and for each attached unit. It is assumed that the request information includes information such as an area, a scale, and a conveyance amount of the facility. By analyzing these, the matching processing unit 104 estimates the type, the number, and the like of required robots and attached units. In these estimations, the matching processing unit 104 may use a machine-learning model such as DNN (Deep Neural Network) or an AI.

Further, the matching processing unit 104 may calculate a matching score indicating a degree of matching between the request information and the supply information. For example, if the applicant U1 has entered a plurality of requests, the matching processing unit 104 may add the numbers of matching items to calculate a matching score. Alternatively, the matching processing unit 104 may calculate the matching score by weighting and adding the matched items using a weight based on a priority or the like.

The communication unit 105 transmits the selected supplier P1 to the applicant U1. For example, the communication unit 105 transmits the contact destination of the supplier P1 to the applicant U1. When a plurality of suppliers is selected, the communication unit 105 may transmit a list of suppliers. Further, the information of the supplier is transmitted to the applicant U1 together with additional information such as the matching score. This allows the applicant U1 to contact the appropriate supplier P1. Alternatively, when the approval of the applicant U1 is obtained, the communication unit 105 may transmit the information of the introduction-desired person to the selected supplier. Of course, the communication unit 105 may transmit the respective contact destinations to both the applicant U1 and the supplier P1. That is, the communication unit 105 may notify at least one of the applicant U1 and the supplier P1 of the matching result.

In this way, the matching device 100 can appropriately match the applicant U1 and the supplier P1. Accordingly, the matching device 100 can introduce the requesting supplier P1 to the applicant U1. Therefore, the applicant U1 can easily introduce the robots, and thus the convenience can be improved. Robot introduction to the facility can be facilitated. The matching device 100 may generate the matching using a machine learning model such as deep learning.

Further, the applicant U1 may evaluate the supplier P1. For example, the applicant U1 uploads the assessment for the introduced robots to the matching site. The supplier P1 may evaluate the applicant U1. For example, the applicant U1 uploads the assessment for the target site to the matching site. Thus, the third party can confirm the evaluation of the past robot introduction. These evaluations may be input in five levels of scores or the like. Alternatively, the applicant U1 or supplier P1 may evaluate by entering a comment, such as textual comment. At least one of the applicant U1 and the supplier P1 inputs a score that evaluates the other, so that the other person can confirm the score.

In addition, the matching processing unit 104 may analyze the supply information and the requested information to present a combination of the supplier P1 and the applicant U1 that are compatible with each other. For example, when the applicant U1 desires to introduce a robot to a hospital, the matching processing unit 104 selects a supplier that is highly evaluated for supplying the robot to the hospital. Alternatively, when a large amount of conveyance is input as the request information, the matching processing unit 104 selects a robot having a high conveyance result or a supplier of a robot having a large conveyance amount. As described above, the matching processing unit 104 may perform matching by analyzing the evaluation result. Alternatively, the matching device 100 may analyze the request information and the supply information to estimate the number of robots and the attached units to be introduced. For example, the matching device 100 can calculate the number of robots and the like based on the facility type, the facility size, and the service type.

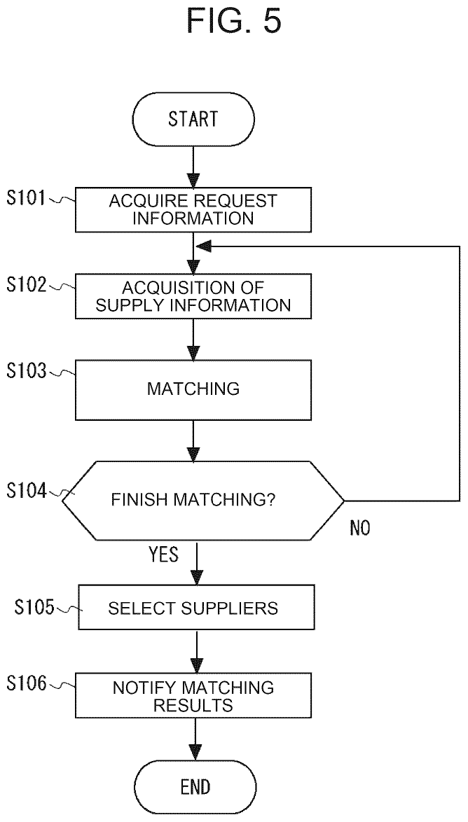

FIG. 5 is a flowchart illustrating an example of a matching method. First, the request information acquisition unit 101 acquires the request information (S101). The request information acquisition unit 101 receives request information from the applicant terminal 400. Next, the supply information acquisition unit 102 acquires the supply information (S102). For example, the supply information acquisition unit 102 reads the supply information stored in the supply information storage unit 103. Here, the supply information acquisition unit 102 sequentially reads the supply information of each supplier.

The matching processing unit 104 performs matching based on the requested information and the supply information (S103). The matching processing unit 104 compares the request information with the supply information, and outputs a value indicating whether or not matching is possible. Alternatively, the matching processing unit 104 compares the request information with the supply information, and outputs a matching score. Further, the matching processing unit 104 may output a matching result based on various requests of the applicant U1.

Next, the matching processing unit 104 determines whether or not matching for all suppliers has been completed (S104). When the matching for all the suppliers has not been completed (NO in S104), the supply information acquisition unit 102 reads the supply information of the subsequent supplier from the supply information storage unit 103 (S102). The matching processing unit 104 performs matching based on the supply information and the requested information (S103).

When the matching for the supply information of all the suppliers is completed (YES of S104), the matching processing unit 104 selects the supplier based on the matching result (S105). For example, the matching processing unit 104 selects the supplier having the highest matching score. The matching processing unit 104 may select two or more suppliers.

The communication unit 105 notifies the applicant U1 of the selected supplier (S106). For example, the communication unit 105 transmits the telephone number, the home page address, the mail address, and the contact information of the supplier to the applicant U1. Of course, the communication unit 105 may transmit the information of the applicant U1 to the supplier P1. In this way, the applicant U1 can contact the appropriate supplier. Further, the supplier P1 may be sent a request information. As a result, the matching device 100 can appropriately perform matching.

In addition, some or all of the processing in the matching device 100, the applicant terminal 400, the supplier terminal 500, and the like described above can be realized as a computer program. The program as described above is stored using various types of non-transitory computer-readable media, and can be supplied to a computer. The non-transitory computer-readable media include various types of tangible recording media. Examples of the non-transitory computer-readable media include magnetic recording media (e.g. flexible disks, magnetic tapes, hard disk drives), magneto-optical recording media (e.g. magneto-optical disks), compact disc read-only memory (CD-ROM), compact disc recordable (CD-R), compact disc rewritable (CD-R/W), and semiconductor memory (e.g. mask ROM, programmable ROM (PROM), erasable PROM (EPROM), flash ROM, random access memory (RAM)). Further, the program may also be supplied to the computer by various types of transitory computer-readable media. Examples of the transitory computer-readable media include electrical signals, optical signals, and electromagnetic waves. The transitory computer-readable media can supply the program to the computer via a wired communication path such as an electric wire and an optical fiber, or a wireless communication path.

The present disclosure is not limited to the above embodiment, and can be appropriately modified without departing from the spirit.

What is claimed is:

1. A matching method that matches an introduction-desired person of a robot and a supplier of the robot by using a computer, the matching method comprising:
obtaining request information related to a robot introduction from the introduction-desired person;
obtaining supply information related to the robot, the supply information being suppliable by the supplier;
performing matching based on the request information and the supply information; and
notifying a matching result to at least one of the introduction-desired person and the supplier.

2. The matching method according to claim 1, wherein at least one of the introduction-desired person and the supplier inputs a score evaluating another one of the introduction-desired person and the supplier, the score being evaluable by the other one of the introduction-desired person and the supplier.

3. The matching method according to claim 1, wherein matching is performed by analyzing the supply information and the request information.

4. A matching system that matches an introduction-desired person of a robot and a supplier of the robot by using a computer, the matching system comprising:
obtaining request information related to a robot introduction from the introduction-desired person;
obtaining supply information related to the robot, the supply information being suppliable by the supplier;
performing matching based on the request information and the supply information; and
notifying a matching result to at least one of the introduction-desired person and the supplier.

5. A non-transitory storage medium storing a program for executing a matching method that matches an introduction-desired person of a robot and a supplier of the robot by using a computer, wherein the matching method includes:
obtaining request information related to a robot introduction from the introduction-desired person;
obtaining supply information related to the robot, the supply information being suppliable by the supplier;
performing matching based on the request information and the supply information; and
notifying a matching result to at least one of the introduction-desired person and the supplier.
